(19) United States
(12) Patent Application Publication
(10) Pub. No.: US 2009/0148393 A1
(43) Pub. Date: Jun. 11, 2009

(54) Multistep Cosmetic Compositions

(75) Inventors: Prithwiraj Maitra, Randolph, NJ (US); Steven Carlo, Monroe, NJ (US)

Correspondence Address: AVON PRODUCTS, INC., AVON PLACE, SUFFERN, NY, 10901, US

(73) Assignee: Avon Products, Inc., New York, NY (US)

(21) Appl. No.: 11/953,915

(22) Filed: Dec. 11, 2007

(51) Int. Cl. A61K 8/19; A61Q 1/02

(52) U.S. Cl. 424/63

(57) ABSTRACT

The present invention relates to a system of cosmetic and dermatological compositions and methods for treating and improving the aesthetic appearance of a biological surface by altering the optical properties of the biological surface, thereby resulting in a natural, flawless appearance. The compositions of the invention may be topically applied to the biological surface, including but not limited to skin, in an amount effective in improving the aesthetic and natural appearance of the biological surface.

### FIELD OF THE INVENTION

This invention generally relates to a system of multiple cosmetic, dermatological, or pharmaceutical compositions and their use, and more particularly to a system of cosmetic compositions and their use in improving the appearance of biological surfaces.

### BACKGROUND OF THE INVENTION

There is an increasing demand in the cosmetics industry to develop products that may be applied topically to the skin to improve the appearance of skin such that the skin has a natural appearance. Ideally, a biological surface, such as skin, would have a translucent appearance with uniform coloring, a smooth and even surface, with no apparent imperfections. Consumers are interested in mitigating or delaying the dermatological signs of chronologically or hormonally-aged or photo-aged skin, such as fine lines, wrinkles, drying, and sagging skin, and other conditions due to a progressive degradation of the skin matrix, as well as improving the appearance of skin damaged by environmental factors, including sun, pollutants, smoking, etc. Therefore, there is a need for cosmetics that assist in creating a flawless, natural look.

Achieving a perfect, flawless and natural look is difficult because cosmetic ingredients which provide the desired color and coverage, are generally opaque and tend to obscure the vibrant and natural translucency of the skin. Pigments or particles with less opacity are available, but they may not provide enough coverage to mask or camouflage the flaws in or on the biological surface, including skin. Cosmetic ingredients can settle in the fine lines and wrinkles, accentuating the flaws and imperfections on the skin's surface resulting in an uneven and unnatural look. Therefore, there remains a need for cosmetic formulations and preparations that convey the perception that the consumer's skin has fewer wrinkles and imperfections by allowing the optimal levels of refraction and reflection of light and increasing the diffusion of light to accomplish, the foregoing.

Currently there is a technology gap in topical skin and color cosmetic products to efficiently blur line lines and wrinkles, as well as provide the required hiding to cover discolorations, age spots, and blemishes to match skin tone with the natural look of a young skin. Traditional ways of optically reducing lines and wrinkles include creating a blurring or soft focus effect on the skin, or by covering lines and wrinkles with opaque pigments leading to unnatural coating on the face Moreover, when traditional inorganic cosmetic pigments are added along with soft focus fillers, the blurring effect is diminished and the coating produces an opaque, chalky, and unnatural appearance. Traditional one-step foundations typically use high opacity pigments which can lead to an unnatural appearance. Accentuation of lines and wrinkles may be observed due to the migration and deposition of pigments into said lines and wrinkles Two-step foundations using an opaque topcoat which is pigmented along with a basecoat may not adequately reduce lines and wrinkles because the benefits of the basecoat may not be optically realized.

There is a need for a system of cosmetic compositions and method of use thereof to improve the aesthetic appearance of skin. Therefore, safe and effective systems of compositions that provide a translucent appearance with uniform coloring, smooth and even biological surfaces, with no apparent imperfections would be advantageous for the formulation of treatments and products for biological surfaces, including the skin.

### SUMMARY OF THE INVENTION

It is an object of the present invention to provide a system of cosmetic, dermatological, or pharmaceutical compositions and their use. More particularly, it is an object of the present invention to provide a system of cosmetic compositions that serves to match a user's skin tone, cover blemishes, and blur lines and wrinkles.

It is an object of the present invention to provide a system comprised of multiple cosmetic compositions, each of the compositions serving to enhance one or more aspects impacting the appearance of a biological surface. It is a further object of the present invention to provide a method of using the compositions which comprise the cosmetic system in a manner so as to optimize each composition's contribution to improving the appearance of a biological surface.

An object of the present invention is to provide a system of two cosmetic compositions, the first composition being a basecoat and the second composition being a topcoat. It is a further object of the invention to provide a pigmented basecoat and a diffused topcoat. A further object of the invention is to provide a method of using a system of two cosmetic compositions, the method being first applying a basecoat to a biological surface and second applying a topcoat to the basecoat. It is a further object of the invention to provide a method of first applying a pigmented basecoat to a biological surface and second applying a diffused topcoat to the pigmented basecoat.

It is an object of the present invention to provide a system of two cosmetic compositions: the first composition provided to cover blemishes and the second composition to blur lines and wrinkles. It is a further object of the present invention to provide a method of first applying a composition to cover blemishes (basecoat) to a biological surface and second applying a composition to blur lines and wrinkles (topcoat) to the basecoat. It is yet a further object of the present invention that use of the first composition in conjunction with the second composition improve the appearance of a biological surface.

In another object of the invention, a first cosmetic composition and a second cosmetic composition are applied sequentially to a substrate (for example, a biological surface). Specifically, a first cosmetic composition is applied to a biological surface as a basecoat, followed by a second cosmetic composition being applied on top of the first cosmetic composition as a topcoat. As a further object of the invention, the first and second cosmetic compositions each have a different refractive index so as to control light diffusion properties. More specifically, the refractive index of the first cosmetic composition is higher than the refractive index of the second cosmetic composition to obtain optimal lateral light diffusion.

In another object of the invention, a first cosmetic composition (basecoat) has a diffused transmittance value that is different than the diffused transmittance value of a second cosmetic composition (topcoat) so as to control light diffusion properties. More specifically, the first cosmetic composition has a diffused transmittance value less than the diffused transmittance value of the second cosmetic, composition. In yet another object of the invention, the first cosmetic composition (basecoat) has a reflectance value different than the reflectance value of the second cosmetic composition (topcoat) so as to control light diffusion properties. More specifically, the first cosmetic composition (basecoat) has a reflectance value higher than the reflectance value of the second cosmetic composition (topcoat).

A further object of the invention is to provide a system of two cosmetic compositions which produce unique optical effects on skin. In yet another object, a method of improving the aesthetic or natural appearance of a biological surface by sequentially applying to the biological surface two cosmetic compositions in an amount effective to improve the aesthetic or natural appearance of the biological surface. The compositions are applied such that the appearance of dermatological signs of damaged or chronologically or hormonally aged or photo-aged skin, such as fine lines, wrinkles, and sagging skin, surface imperfections, and discoloration is diminished.

These and other objects and advantages of the present invention, and equivalents thereof, are achieved by compositions having a system of two cosmetic compositions, and methods of using such compositions for topical application in order to improve the aesthetic and natural appearance of a biological surface, including for example, skin.

### BRIEF DESCRIPTION OF THE DRAWING

FIG. 1 shows a schematic representation of light diffusion using a one-step foundation on skin.

FIG. 2 shows a schematic representation of light diffusion using a two-step foundation (diffused basecoat and pigmented topcoat) on skin.

FIG. 3 shows a schematic representation of light diffusion using a two-step foundation (pigmented basecoat and diffused topcoat) on skin.

FIG. 4 shows percent diffused transmittance of a pigmented basecoat with diffused topcoat compared with diffused basecoat with pigmented topcoat.

FIG. 5 shows percent reflectance of a pigmented basecoat with diffused topcoat compared with diffused basecoat with pigmented topcoat.

### DETAILED DESCRIPTION OF THE INVENTION

In accordance with the foregoing objectives and others detailed herein, the invention overcomes deficiencies associated with the prior art by providing a system of cosmetic compositions which improves the aesthetic appearance of biological surface resulting from, for example, the chronological aging process, environmental damage to the biological surface, or natural imperfections. The system of cosmetic compositions and methods thereof, once applied to a biological surface or synthetic biological surface, enhances the appearance of the surface by altering the appearance of a biological surface by enhancing diffused transmittance and reflective properties, thereby improving the aesthetic appearance of the biological surface. The aesthetic appearance of the biological surface, including but not limited to skin, keratinous tissues, hair, and nails, may be achieved by topical application of the inventive composition to the biological surface on a daily basis or when a natural appearance is desired.

The inventive system of cosmetic compositions alters the optical properties of a cosmetic composition to provide hiding, opacity, coverage, blurring and natural appearance. The system of cosmetic compositions comprises two cosmetic compositions, the first composition being a basecoat and the second composition being a topcoat. The cosmetic compositions present in the system are suitable for topical skin application. The term “basecoat” as used herein refers to a cosmetic layer that is applied directly to a substrate. Preferably, the substrate is a biological surface, such as skin. The term “topcoat” as used herein refers to a cosmetic layer that is applied on top of a different cosmetic layer. Preferably, the topcoat is applied to the basecoat.

The inventive system of cosmetic compositions provides a pigmented basecoat and a diffused topcoat. A pigmented basecoat is used to match the skin tone and to cover blemishes or age spots. A diffused topcoat is used to blur lines and wrinkles. Application of a pigmented basecoat to a biological surface is followed by application of a diffused topcoat to the basecoat. The topcoat may be comprised of diffusers, film formers, and a cosmetically or pharmaceutical acceptable carrier. The basecoat may be comprised of a pigment and a cosmetically or pharmaceutically acceptable carrier. The basecoat may be a traditional foundation base.

Efficiency of blurring or soft focus effect or the ability to hide lines and wrinkles is believed to be a function of the diffused transmittance parameter (“Quantification of the Soft Focus Effect”, Cosmetics & Toiletries; Vol. 111, July 1996, p. 57) (the disclosure of which is incorporated herein by reference). The references “A Natural-Looking Makeup”, Cosmetics & Toiletries, Vol. 112, 1997, p. 39 and “Measuring Soft Focus Properties of Cosmetic Filler Particles”, HAPPI, August 2003, are also incorporated by reference. A goniospectrophotometer, for example, from Murakami Color Research Laboratory, Model GSP-1B, can be used to measure color as a function of angle in both reflectance as well as transmittance mode. The diffused topcoat may be colorless or may be slightly tinted.

A diffused topcoat may be characterized by a percent diffused transmittance value of 20 or greater, preferably 30 or greater, and more preferably 40 or greater for an approximately 10 micron thin film on a glass plate. In one embodiment, the diffused topcoat has a percent diffused transmittance value of about 55 or greater for an approximately 10 micron thin film. In another embodiment, the diffused topcoat has a percent diffused transmittance value of about 60 or greater for an approximately 10 micron thin film. In another embodiment., the diffused topcoat has a percent diffused transmittance value of about 65 or greater for an approximately 10 micron thin film. In one embodiment, the diffused topcoat has a percent diffused transmittance value of about 64 for an approximately 10 micron thin film. A diffused topcoat may be characterized by a percent, reflectance of 60 or less, preferably 50 or less, and more preferably 40 or less for an approximately 10 micron thin film on a glass plate. In one embodiment, the diffused topcoat has a percent reflectance of about 20 or less for an approximately 10 micron thin film. In another embodiment, the diffused topcoat has a percent reflectance of about 15 or less for an approximately 10 micron thin film. In another embodiment, the diffused topcoat has a percent reflectance of about 10 or less for an approximately 10 micron thin film. In one embodiment, the diffused topcoat has a percent reflectance of about 12 for an approximately 10 micron thin film.

A pigmented basecoat may be characterized by a low percent diffused transmittance value of 50 or less, preferably 40 or less, and more preferably 30 or less for an approximately 10 micron thin film. In one embodiment, the pigmented basecoat has a percent diffused transmittance value of about 30 or less for an approximately 10 micron thin film. In another embodiment, the pigmented basecoat has a percent diffused transmittance value of about 25 or less for an approximately 10 micron thin film. In another embodiment, the pigmented basecoat has a percent diffused transmittance value of about 20 or less for an approximately 10 micron thin film. In one embodiment, the pigmented basecoat has a percent diffused transmittance value of about 25 for an approximately 10 micron thin film. A pigmented basecoat may be characterized by a percent reflectance of 10 or greater, more preferably 20 or greater for an approximately 10 micron thin film on a glass plate. In one embodiment, the pigmented basecoat has a percent reflectance of about 15 or greater for an approximately 10 micron thin film. In one embodiment, the pigmented basecoat has a percent reflectance of about 20 or greater for an approximately 10 micron thin film. In one embodiment, the pigmented basecoat has a percent reflectance of about 25 or greater for an approximately 10 micron thin film. In one embodiment, the pigmented basecoat has a percent reflectance of about 30 or greater for an approximately 10 micron thin film, in one embodiment, the pigmented basecoat has a percent reflectance of about 25 for an approximately 10 micron thin film.

In the inventive cosmetic system having a pigmented basecoat with a diffused topcoat, if is desirable to have a topcoat with a high percent diffused transmittance and a basecoat with a high percent reflectance as explained above. The inventive cosmetic system may be characterized by a percent diffused transmittance value of about 10 or greater, more preferably about 20 or greater, and more preferably about 30 or greater for an approximately 10 micron thin film of diffused topcoat applied to an approximately 10 micron thin film of pigmented basecoat. The inventive cosmetic system may be characterized by a percent, reflectance of about 10 or greater for an approximately 10 micron thin film of diffused topcoat applied to an approximately 10 micron thin film of pigmented basecoat. In one embodiment, the inventive cosmetic system has a percent diffused transmittance of about 15 and a percent reflectance value of about 75 for an approximately 10 micron thin film of diffused topcoat applied to an approximately 10 micron thin film of pigmented basecoat. In one embodiment, the inventive cosmetic system has a percent diffused transmittance of about 25 and a percent reflectance value of about 25 for an approximately 10 micron thin film of diffused topcoat applied to an approximately 10 micron thin film of pigmented basecoat.

The diffused topcoat and pigmented basecoat will each have an index of refraction. Preferably, the refractive index of the diffused topcoat is less than the refractive index of the pigmented basecoat to obtain optimal light diffusion. The refractive indices of various materials may be evaluated using a refractometer. Details with respect to the principles of refraction can be found in Optics by Eugene Hecht (Fourth Edition), 2002. Details with respect to refractive indices of materials can be found in the CRC Handbook of Chemistry and Physics, 86th Edition, 2005-2006, which is herein incorporated by reference in its entirety.

The refractive index of the pigmented basecoat can be from about 1.38 to about 3.52; more preferably about 1.40 to about 3.50; more preferably about 1.42 to about 3.40. In one embodiment the pigmented basecoat has a refractive index of between about 1.42 and about 1.60. Pigments in this refractive index range include, but are not limited to, titanium dioxide (rutile or anatase), zinc oxide and iron oxide. In one embodiment of the invention, the pigment material in the basecoat is titanium dioxide (anatase) with an index of refraction of about 2.50.

The refractive index of the diffused topcoat can be from about 1.30 to about 2.20; more preferably about 1.40 to about 2.00. In one embodiment of the invention, the diffused topcoat has an index of refraction of about 1.50. In another embodiment, the diffused topcoat incorporates nylon having a refractive index of about 1.53. In another embodiment, the diffused topcoat incorporates light diffusing particles made of silica beads having a refractive index of about 1.46.

Suitable inorganic pigments include, but are not limited to, titanium dioxide, zirconium oxide and cerium oxide, as well as zinc oxide, iron oxide, chromium oxide and ferric blue. Suitable organic pigments include, but are not limited to barium, strontium, calcium, and aluminum lakes and carbon black. Any pigment material of the inventive composition producing the desired effects may be used, non-limiting examples of which include a metal oxide, for example, titanium dioxide, iron oxide, and aluminum oxide. For typical pigments used in cosmetic industry, refer to the Cosmetic Ingredient Dictionary (ICID) and Handbook, 10th Edition (2004), published by the Cosmetic, Toiletry, and Fragrance Association (CTFA).

Suitable light diffusers for use in a diffused topcoat include, but are not limited to, nylon, polyurethane, silica beads, Tospearl (Momentive), silicone crosspolymers such as Velvesil (Momentive), poly (methyl methacrylate) (PMMA), polyethylene (PE), polystyrene (PS), polytetrafluoroethylene (PTFE, e.g., Teflon®), polymethylsiloxane, cellulose beads, boron nitride, mica, polyurethane powder, sericite, silica, firmed silica, fumed alumina, talc, titanium dioxide, zinc oxide, iron oxide, silicone powder and combinations thereof. Preferable light diffusers for use in a diffused topcoat include nylon, polyurethane, silica beads, Tospearl (Momentive), silicone elastomers such as Velvesil (Momentive), poly (methyl methacrylate) (PMMA), polyethylene (PE), polystyrene (PS), polytetrafluoroethylene (PTFE, e.g., Teflon®), polymethylsiloxane, cellulose beads, boron nitride, mica, polyurethane powder, sericite, silica, fumed silica, fumed alumina, silicone powder and combinations thereof. The light diffuser is preferably present from about 0.01 weight % to about 50 weight % of the total weight of the composition.

Suitable film formers for use in a diffused topcoat include, but are not limited to, sulfopolyester resins, polyvinylacetate, polyvinyl alcohol polymers, acrylic resins, silicone acrylate polymers (such as those available from Shin Etsu), polyvinylpyrrolidones, high molecular weight silicones, organosiloxanes, polyurethanes, hydrophobic acrylate copolymers, as well as others known in the art (for example those listed in WO 03/105790, incorporated herein). The film former is preferably present from about 0.01. weight % to about 20 weight % of the total weight of the composition.

Suitable cosmetically acceptable carriers for use in a diffused topcoat include, but are not limited to, emulsions, gels, creams, lotions and others known in the art as described further below. The topcoat may be anhydrous (hydrocarbon or silicone based) or may be an emulsion.

In one embodiment of the invention, the pigmented basecoat is applied directly to a biological surface. Following the application of a pigmented basecoat to a biological surface, a diffused topcoat is applied on top of the pigmented basecoat. Preferably, the pigmented basecoat and diffused topcoat are compatible with each other.

The basecoat and topcoat are each capable of altering scattering behavior of visible light, thereby providing soft focus or blurring effects useful in cosmetic compositions which camouflage wrinkles, fine lines, or imperfections of biological surfaces. The difference between the refractive indices of the basecoat and topcoat may range from about 0.0.1 to about 1.50. In one embodiment of the invention, the difference between the refractive indices of the basecoat and topcoat is less than about 1.0. In one embodiment, the difference between the refractive indices of the basecoat and topcoat is about 0.2. In one embodiment, the basecoat is pigmented to match the skin tone and cover blemishes. The topcoat may be colorless or may be slightly tinted to blur lines and wrinkles. Preferably, a pigmented basecoat is provided to match skin tone and cover blemishes and a colorless topcoat is provided to blur lines and wrinkles.

A basecoat may be applied to skin as a layer in amount having a thickness of between approximately 1.0 and approximately 20 microns. Preferably, the basecoat is a layer that is approximately 10 microns in thickness. A topcoat may be applied to a basecoat as a layer in amount having a thickness of between approximately 1.0 and approximately 20 microns. Preferably, the topcoat is a layer that is approximately 10 microns in thickness. Preferably, the total thickness of the topcoat and basecoat combined is less than approximately 20 microns, more preferably between 2.0 and 20 microns in thickness. In one embodiment, the topcoat is applied in an amount that is approximately 2.0 mg per square centimeter. In one embodiment, the basecoat is applied in an amount that is approximately 2.0 mg per square centimeter.

In one embodiment of the invention, a cosmetic system comprising a first cosmetic composition and a second cosmetic composition is provided. Preferably, the cosmetic system is provided as a cosmetic kit comprising two compositions. In one embodiment, a method of improving the aesthetic or natural appearance of a biological surface by sequentially applying to the biological surface two cosmetic compositions in an amount effective to improve the aesthetic or natural appearance of the biological surface.

In one embodiment a method of applying a pigmented basecoat to a biological surface is followed sequentially by applying a diffused topcoat, to the pigmented basecoat. Preferably, a diffused topcoat is applied to a pigmented basecoat less than approximately 10 minutes after a pigmented basecoat is applied to a biological surface; more preferably, a diffused topcoat is applied to a pigmented basecoat less than approximately 7 minutes after a pigmented basecoat is applied to a biological surface; most preferably, a diffused topcoat is applied to a pigmented basecoat less than approximately 5 minutes after a pigmented basecoat is applied to a biological surface.

In alternative embodiments, three or more compositions are provided. In one such embodiment, a system of three cosmetic compositions is provided. In a system having three cosmetic compositions, a first composition is provided having a pigment, a second composition is provided having a diffuser, and a third composition is provided. In embodiments having three or more compositions, the third composition may be provided to impart, cosmetic benefits. Beneficial agents for use in the third composition may include, but are not limited to, absorbents, anti-acne actives, hydrophilic or hydrophobic conditioners, skin soothers, sunscreen actives, vitamins, or combinations thereof.

The third composition is provided for application to one or more surfaces, for example, application to a substrate such as skin, or application to a layer of the second composition, or application to a layer of the first composition. Preferably, the third composition is applied as a layer to a biological surface, followed by application of a layer of the first composition having a pigment, followed by application of a layer of the second composition having a diffuser. In one embodiment, a third composition having an absorbent is applied as a layer to the skin, followed by application of a first composition having a pigment, followed by application of a second composition having a diffuser.

In an alternative embodiment, a third composition is provided for application as a topmost layer above both a pigmented base layer and an intermediate layer having a diffuser. Specifically, the first composition having a pigment is applied as a layer to a biological surface, followed by a layer of the second composition having a diffuser, followed by a layer of the third composition. In such an embodiment, the third composition may be provided having beneficial agents such as a sunscreen or a sealant.

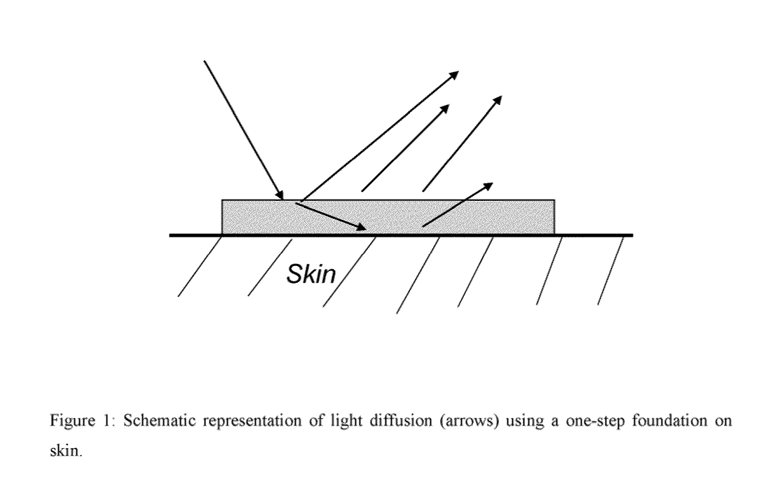

FIG. 1 shows a schematic representation of light diffusion using a one-step pigmented foundation on the surface of skin. Light directed towards the pigmented foundation and skin (represented by arrow) interacts with the pigmented foundation. Light is shown diffusing info the pigmented foundation layer and reflecting from the pigmented foundation and skin.

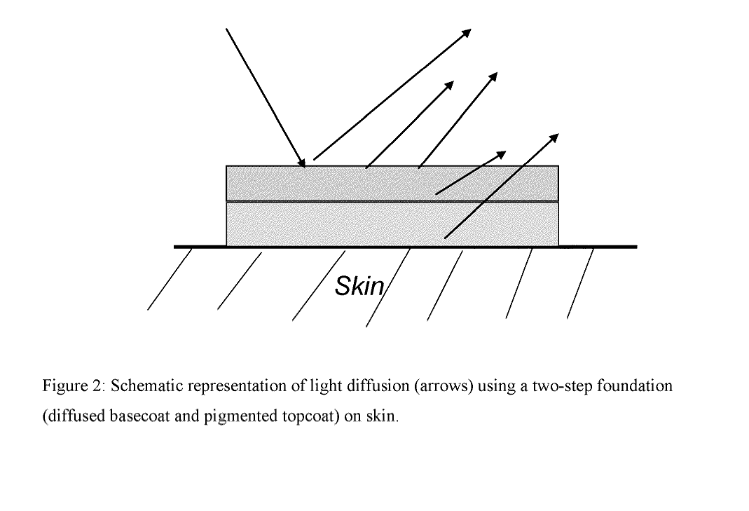

FIG. 2 shows a schematic representation of light diffusion using a two-step foundation on the surface of skin. Two layers are shown, a basecoat on top of the skin, and a topcoat on top of the basecoat. A diffused basecoat and pigmented topcoat are shown in FIG. 2. Light directed towards the topcoat, basecoat, and skin (represented by arrow) interacts with the pigmented topcoat and diffused basecoat. Light is shown diffusing into the pigmented foundation layer and reflecting from the pigmented topcoat, from the diffuse basecoat, and skin.

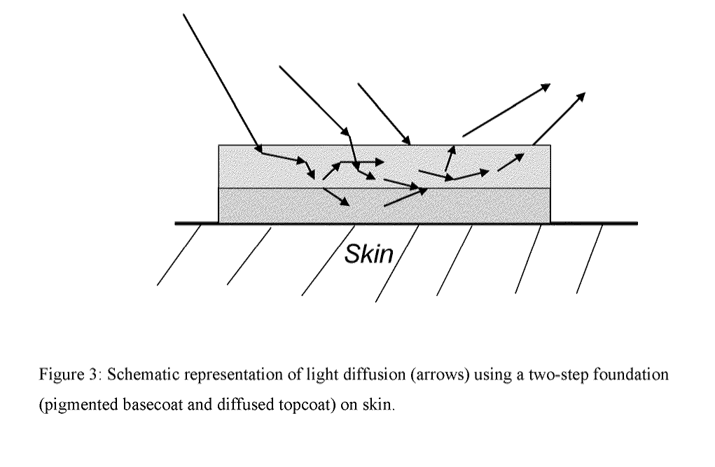

FIG. 3 shows a schematic representation of light diffusion using a two-step foundation of the invention on the surface of skin. Two layers are shown in FIG. 3, a basecoat on top of the skin, and a topcoat on top of the basecoat. A pigmented basecoat and diffused topcoat are shown in FIG. 3, light directed towards the topcoat, basecoat, and skin (represented by arrow) interacts with the diffused topcoat and pigmented basecoat. Light is shown diffusing into the diffused topcoat and scattering. Light is reflected from the pigmented basecoat, from the diffuse topcoat and skin. The amount of light reflected from the two-step foundation system of FIG. 3 is less than that reflected in the inverted two-step foundation system of FIG. 2.

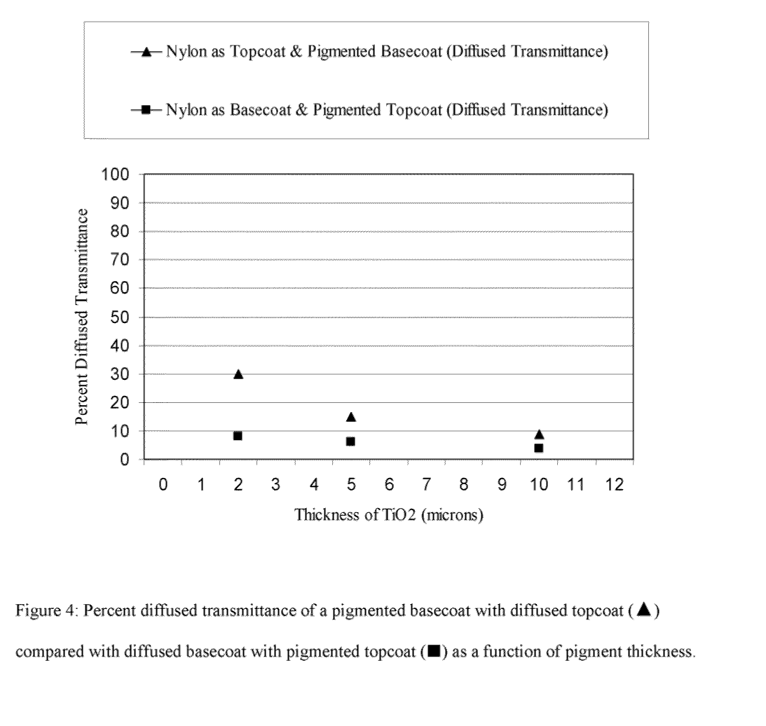

FIG. 4 shows percent diffused transmittance of a system having a pigmented basecoat with a diffused topcoat of the invention (▴) compared with a cosmetic system having a diffused basecoat with a pigmented topcoat (▪) as a function of pigment thickness. For each measured thickness, the system having a pigmented basecoat with diffused topcoat of the invention exhibits a greater diffused transmittance value than a system having a diffused basecoat with a pigmented topcoat. For each of the two systems (pigmented basecoat with a diffused topcoat of the invention (▴) and diffused basecoat with a pigmented topcoat (▪), the diffused topcoat or diffused basecoat had a thickness of about 10 microns.

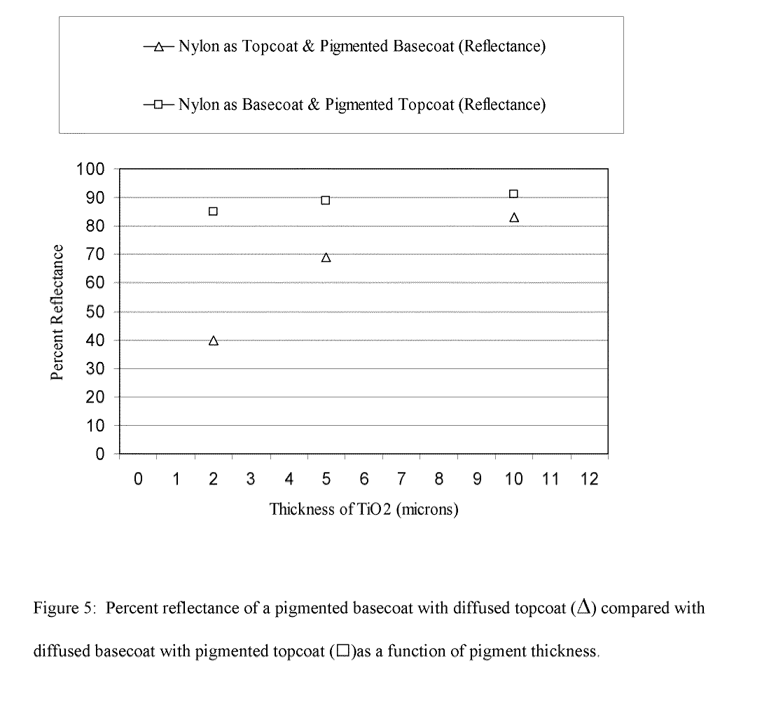

FIG. 5 shows percent reflectance of a system having a pigmented basecoat with a diffused topcoat of the invention (Δ) compared with a cosmetic system having a diffused basecoat with a pigmented topcoat (□) as a function of pigment thickness. For each measured thickness, the system having a pigmented basecoat with diffused topcoat of the invention exhibits a lower reflectance value than a system having a diffused basecoat with a pigmented topcoat. For each of the two systems (pigmented basecoat with a diffused topcoat of the invention (Δ) and diffused basecoat with a pigmented topcoat (□), the diffused topcoat or diffused basecoat had a thickness of about 10 microns.

FIGS. 4 and 5 present data exemplifying the properties described in FIGS. 2 and 3. Specifically, an inventive cosmetic system of FIG. 3 (pigmented basecoat and diffused topcoat) graphically depicts greater diffused transmittance (more blurring) than the system of FIG. 2 (diffused basecoat and pigmented topcoat), as is shown in the data FIG. 4. Similarly, an inventive cosmetic system of FIG. 3 (pigmented basecoat and diffused topcoat) graphically depicts lower reflectance than the system of FIG. 2 (diffused basecoat and pigmented topcoat), as is shown in the data of FIG. 5.

FIG. 2 (diffused basecoat and pigmented topcoat) and FIG. 5 indicate that a majority of the light directed towards the skin's surface is reflected off the pigmented topcoat and does not even reach the diffused basecoat. For this reason, the benefit of the diffused basecoat is not realized. In contrast, use of a diffused topcoat and pigmented basecoat in a cosmetic system of the invention as depicted in FIG. 3, serves to scatter light to reduce the appearance of lines and wrinkles while maintaining skin tone.

The cosmetic system of the invention manipulates light diffusion properties through a diffused topcoat and a pigmented basecoat on skin. The diffused topcoat has a lower refractive index than the pigmented basecoat. Light, for example sunlight, first enters the diffused topcoat, then the pigmented basecoat. The difference in refractive indices between the topcoat and the basecoat will control the total reflection and scattering of light at the interface between the topcoat and basecoat. Design and choice of diffused topcoat and pigmented basecoat can increase the angle of incidence greater than the critical angle for total internal reflection, thus enhancing the light diffusion along the interface. By using a high refractive index pigmented basecoat, one can obtain high coverage along with a binning effect due to the topcoat. A high index of refraction basecoat with a low index of refraction topcoat enable the cosmetic system to exhibit a blurring effect by enhancing diffused transmittance and thereby improving the appearance of the biological surface onto which the compositions were applied. Without wishing to be bound by theory, the efficiency of the blurring and soft focus effect or the ability to camouflage fine lines and wrinkles is believed to be a function of the diffused transmittance parameter (“Quantification of the Soft Focus Effect”, Cosmetics & Toiletries, 111:57, 1996). Therefore, a system of compositions that produces high diffused transmittance would be beneficial for producing a blurring effect, improving the natural appearance of the biological surface to which the cosmetic is applied, and overcoming the drawbacks of typical one-step cosmetics that are opaque, unnatural and chalky, and two-step cosmetics involving a diffused basecoat and pigmented topcoat.

The advantages of the inventive cosmetic system include, but are not limited to, delivering flawless finish on the skin by eliminating both the textural as well as color imperfections. Another advantage of a two-step cosmetic composition system is the optimization of the benefits of each composition (for example, blurring and coverage) to enhance the system's overall performance. Another advantage of a two-step cosmetic composition system is tailoring the composition to provide films and coatings that may provide ultraviolet (UV) protection. Another advantage of a two-step cosmetic composition system is tailoring the basecoat so that it may absorb oil, sebum, and moisture, for example. These properties provide additional benefits in cosmetic and dermatological formulations or compositions, and enhance the aesthetic and natural appearance of biological surfaces, such as skin.

A further embodiment of the invention provides a two-step cosmetic composition system that has optical properties which enhance the aesthetic and natural appearance of a biological surface resulting in optical blurring and diffused light transmittance, light scattering and reflection, thereby reducing the appearance of dermatological signs of chronological aging, photo-aging, hormonal aging, and/or actinic aging; reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; reducing the appearance and/or depth of lines and/or wrinkles; improving the appearance of suborbital lines and/or periorbital lines; reducing the appearance of crow's feet; improving the appearance of rejuvenating and/or revitalizing skin, decreasing the appearance of aging skin; reducing the appearance of skin fragility, reducing the appearance of a loss of glycosaminogiycans and/or collagen; reducing the appearance of estrogen imbalance; reducing the appearance of skin atrophy; reducing the appearance of hyperpigmentation; reducing the appearance of skin discoloration; improving the appearance of skin tone, radiance, clarity and/or tautness; reducing the appearance of sagging skin; improving the appearance of skin firmness, plumpness, suppleness and/or softness; improving the appearance of procollagen and/or collagen production; improving the appearance of skin texture and/or retexturization; improving the appearance of skin barrier repair and/or function; improving the appearance of skin contours; improving the appearance of decreased skin luster and/or brightness; improving the appearance of dermatological signs of fatigue and/or stress; improving the appearance of environmental stress; improving the appearance of cellular aging; improving the appearance of skin dehydration; improving the appearance of elastic and/or resilient skin, improving the appearance of microcirculation; decreasing the appearance of cellulite formation; or any combinations thereof,

Another embodiment of the invention relates to a method of improving the aesthetic or natural appearance of a biological surface comprising applying to the biological surface, including but not limited to, keratinous tissue, skin, hair and nails, a pigmented basecoat followed by applying to the basecoat a diffused topcoat, each in an amount effective to improve the aesthetic or natural appearance of the biological surface.

The biological surface may be any surface to which cosmetics, personal care products, dermtatological, and pharmaceutical compositions are typically applied, including but not limited to skin, lips, hair, and nails. The system of the invention that is applied to biological surfaces improves or enhances the aesthetic appearance of skin by camouflaging the natural aging process, discoloration, chronic and cumulative damage to biological surfaces, and imperfections on the surface.

Embodiments of the invention relate to the discovery that a system of cosmetic compositions camouflages fine lines, wrinkles, pores, sagging, or other conditions due to aging. Thus, by relying on the optical properties of each composition and the interface between each composition enables one to camouflage the signs of aging, dermatological degeneration of biological surfaces, and imperfections of biological, surfaces, thereby improving the aesthetic and natural appearance of skin.

One embodiment of the invention relates to methods of applying the system of cosmetic compositions to an affected area of the skin. The system is preferably applied topically at least once daily, preferably twice a day, and remains on the affected area of the skin, where the affected area of the skin includes, but is not limited to, the lace, neck, legs and thighs, scalp, and overall body.

The system of cosmetic compositions is useful in improving the natural and aesthetic appearance of a biological surface, including skin, lips, hair, and nails when applied topically. The compositions of the invention may also include, non-limiting examples of active ingredients useful in reducing, diminishing, or camouflaging medical and/or cosmetic conditions associated with aging, inflammation, and degeneration of the biological surface. Such conditions, as used herein, commonly include, but are not limited to, dermatological aging (chronological aging, hormonal aging and/or actinic aging), dermatitis, skin and hair fragility, hirsutism, rosacea, skin blemishes, sensitive skin, hyperpigmentation or hypopigmentation, thinning skin, roughness, keratosis, skin atrophy, wrinkles, lines, hyperplasia, fibrosis, and any combinations thereof. The active components of the present invention may also be useful in enhancing the general health, vitality, condition, and aesthetic appearance of the skin.

In accordance with the invention, the system of cosmetic compositions may be useful in topically applied formulations, antioxidants, antiinflammatories, sunscreens, cosmetics, including makeup, and formulations for reducing dermatolosical signs of aging, including wrinkles, fine lines, and sagging skin, and the like. Also in accordance with this invention, additional constituents in a composition may be formulated in a variety of product forms. The compositions may be prepared in targeted delivery systems, e.g. creams, lotions, moisturizers, gels, toners, serums, sprays, foams, powders, and the like, particularly for topical application and administration.

The present invention further provides the system of cosmetic compositions, preferably for topical administration or for targeted delivery without inducing significant irritation. The inventive system, of compositions is suitable for all skin types, such as sensitive, normal, dry, or oily, preferably sensitive to dry skin, as well as mature skin. In particular embodiments, the compositions may be suitable for dry skin. The system of compositions is applied to the skin for a period of time sufficient to enhance the natural and aesthetic appearance of skin. The system of compositions may be applied topically once, twice, or more daily to biological surfaces, including but not limited to skin, lips, and hair.

The topical compositions may be formulated into liposomes which may comprise other additives or substances, and/or which may be modified to more specifically reach or remain at a site following administration. The compositions of embodiments of the present invention yield improvements to the aesthetic appearance of skin by camouflaging or improving upon at least one of the previously described conditions, or combinations thereof.

As will be appreciated by the practitioner, cosmetic treatments comprising a system of cosmetic compositions may be carried out, for example, by topically applying the system of cosmetic compositions as described herein according to the routine technique for administering such system. The topical system of cosmetic or dermatological, or pharmaceutical compositions preferably is applied once daily for a period of at least one week, but may include a period of about 2, 4, 8, 12 or more weeks. The system of cosmetic composition is preferably applied to the face and neck, but may be applied to any area of skin in need of aesthetic improvement, where the cosmetic compositions remain on the affected area of skin, and preferably not removed or rinsed off the skin. Routine and commonly practiced techniques encompass the application of creams, lotions, gels, sera, ointments, makeup, sunscreen compositions, or the like, to the skin. Preferably the system of cosmetic compositions is a topical leave-on formulation, where spraying as a form of application is also envisioned.

Another embodiment of the invention encompasses systems of cosmetically, dermatologically, or pharmaceutically acceptable compositions which are suitable for contact with living mammalian tissue, including human tissue, or synthetic equivalents thereof, with virtually no adverse physiological effect to the user. Compositions embraced by this invention can be provided in any cosmetically and/or dermatologically suitable form, preferably as a lotion or cream, but also in an anhydrous or aqueous base, as well as in a sprayable liquid form. Other suitable cosmetic product forms for the compositions of this invention include but are not limited to, for example, an emulsion, a cream, a balm, a gloss, a lotion, a foam, a mask, a serum, a toner, an ointment, a mousse, a patch, a pomade, a solution, a spray, a wax-based stick, or a towelette. In addition, the compositions contemplated by this invention can include one or more compatible cosmetically acceptable adjuvants commonly used and known by the skilled practitioner, such as fragrances, emollients, humectants, preservatives, vitamins, chelators, thickeners, perilla oil or perilla seed oil (WO 01/66067 to a “Method of Treating a Skin Condition,” incorporated herein) and the like, as well as other botanicals such as aloe, chamomile, and the like, and as further described below.

The cosmetic compositions of the present invention may be contained in a cosmetically, dermatologically, physiologically, and pharmaceutically acceptable vehicle, medium, diluent or carrier, for use in reducing, ameliorating, or preventing the dermatological signs associated with aging and inflammation of biological surfaces. In an embodiment embracing topical applications, the compositions of this invention comprise a medium (vehicle, diluent or carrier) that is compatible with mammalian biological surfaces, including skin, lips, hair and nails. The compositions can be formulated as an aqueous phase, an oil phase, alcohol, or aqueous/alcohol-based solutions, ointments, creams, lotions, gels, a wax-in-water emulsion, or water-in-oil, oil-in-water, of water-oil-water triple emulsions having the appearance of a cream or gel, microemulsions, or aerosols.

The aqueous phase is a mixture of one or more water soluble or water dispersible ingredient, which can be liquid, semi-solid or solid at room temperature (25° C.). The vehicle comprises or can be in the form of a suspension, dispersion or solution in water or an aqueous-alcoholic vehicle, which may contain a thickener or gellant. A person skilled in the art can select the appropriate cosmetic form, the ingredients contained therein, as well as the method for preparing it, on the basis of the knowledge that the skilled artisan possesses.

In one embodiment, one or both of the compositions may include an aqueous phase which may contain water or a mixture of water and at least one hydrophilic organic solvent in particular an alcohol, especially a linear or branched lower monoalcohol containing from 2 to 5 carbon atoms, e.g., ethanol or propanol; a polyol, e.g., propylene glycol, sorbitol, glycerol, diglycerol, panthenol, or polyethylene glycol, and mixtures thereof. This aqueous phase may represent from about 0.5 weight % to about 99.99 weight %, based upon the total weight of the composition.

In another embodiment when one or more compositions in the system of the invention is in the form of an emulsion, one or more compositions may also optionally comprise a surfactant, preferably in an amount of from about 0.1 weight % to about 30 weight %, and in particular, from about 1 weight % to about 20 weight %, based upon the total weight of the composition.

In a further embodiment of the invention, one or more compositions may also comprise a thickening polymer such as an amphophilic polyurethane, a polyacrylic homopolymer or copolymer, a polyester, or a hydrocarbon-based resin. Other non-limiting polymers include, homopolymers or copolymers of: vinyl esters of an aliphatic aid having 1 to 18 carbon atoms, such as vinyl acetate; acrylic acid esters and methacrylic acid esters of an alcohol having 1 to 18 carbon atoms, such as methyl acrylate, ethyl acrylate, butyl acrylate, 2-ethylhexyl acrylate, methyl methacrylate, ethyl methacrylate and butyl methacrylate; and mono and di-ethylenically unsaturated hydrocarbons, including ethylene iso-butylene, styrene and aliphatic dunes, including butadiene, isoprene and chloroprene.

One embodiment of the invention further relates to one or more compositions of the invention which may also comprise an oil phase containing oil soluble or oil dispersible ingredients that are liquid at room temperature (25° C.) and/or oily or waxy substances that are solid at room temperature, such as waxes, semi-solids, gums, and mixtures thereof. This oily phase may also contain organic solvents.

Suitable oily materials that are liquid at room temperature, often referred to as oils, include hydrocarbon-based oils of animal origin such as perhydrosqualene; hydrocarbon-based plant, oils such as liquid triglycerides of fatty acids of 4 to 10 carbon atoms, for instance, heptanoic or octanoic acid triglycerides, or oils such as sunflower oil, corn oil, soybean oil, grapeseed oil, castor oil, avocado oil, caprylic/capric acid triglycerides, jojoba oil; linear or branched hydrocarbons of mineral or synthetic origin such as liquid paraffins and derivatives thereof, petroleum jelly; synthetic esters and ethers, in particular esters of fatty alcohols, namely; for example, isopropyl myristate, 2-ethylhexyl palmitate, 2-octyldodecyl stearate, isostearyl isostearate; hydroxylated esters such as isostearyl lactate, octyl hydroxystearate, octyldodecyl hydroxystearate, heptanoates, octanoates and decanoates of fatty alcohols; polyol esters such as propylene glycol dioctanoate, neopentyl glycol diheptanoate, diethylene glycol diisononanoate, and pentaerytritol esters; fatty alcohols containing from 12 to 26 carbon atoms such as octyldodecanol, 2-butyloctanol, 2-hexyldecanol, 2-undecylpentadecanol, oleyl alcohol; partially hydrocarbon-based fluoro oils and/or fluorosilicone oils; silicone oils such as volatile or non-volatile, linear or cyclic polydimethylsiloxanes (PDMS) that are liquid or semisolid at room temperature such as cyclomethicones and dimethicones, optionally comprising a phenyl group, for instance phenyl trimethicones, siloxanes, and mixtures thereof. These oils are usually present in an amount of about 0 weight % to about 90 weight %, preferably from about 1.0 weight % to about 80 weight % by weight of the oil phase.

The oil phase of one or more compositions of the invention may also comprise one or more cosmetically acceptable organic solvents. These solvents are present in an amount of about 0.1 weight % to about 80 weight %, preferably about 1.0 weight % to about 50 weight %, based on the total weight of the composition, and may be selected from the group consisting of lipophilic organic solvents, amphophilic organic solvents and mixtures thereof. Suitable solvents which may be used in the composition of the invention include acetic acid esters such as methyl, ethyl, butyl, amyl or 2-methoxyethyl acetate; isopropyl acetate; hydrocarbons such as toluene, xylene, p-xylene, hexane or heptane, ethers containing at least 3 carbon atoms, and mixtures thereof.

One or more compositions of the invention may further comprise any ingredient conventionally used in the cosmetics field. These ingredients include preserving agents, aqueous phase thickeners (polysaccharide biopolymers, synthetic polymers) and fatty-phase thickeners, fragrances, hydrophilic and lipophilic active agents, and mixtures thereof. The amounts of these various ingredients are those conventionally used in the cosmetics field to achieve their intended purpose, and range typically from about 0.1. weight % to about 20 weight %, based upon the total weight of the composition. The nature of these ingredients and their amounts must be compatible with the production of the compositions of the invention.

One or more compositions of the invention may also comprise an additional particulate phase, typically present in an amount, of about 0.1 weight % to about 30 weight %, based upon the total weight of the composition, preferably from about 0.5 weight % to about 20 weight %, and which can comprise pearlescent agents and/or fillers used in cosmetic compositions. Suitable pearlescent agents include mica coated with titanium dioxide or with iron oxide.

Fillers are normally present in an amount of about 0.1 weight % to about 30 weight %, based on the total weight, of the composition, preferably about 0.5 weight % to about 15 weight %. Suitable fillers include talc, silica, zinc stearate, mica, kaolin, nylon (in particular orgasol) powder, polyethylene powder, Teflon®, starch, boron nitride, copolymer microspheres such as Expancel® (Nobel Industrie; Sweden), Polytrap® (Dow Corning, Inc.; Midland, Mich.), and silicone resin microbeads (Tospearl®; GE Toshiba Silicones; Japan).

The oil phase of the compositions of the invention may comprise one or more waxes, gums, or mixtures thereof. The waxes include hydrocarbon-based waxes, fluoro waxes and/or silicone waxes and can be of plant, mineral, animal and/or synthetic origin. In particular, the waxes have a melting point of greater than 25° C., preferably greater than 45° C. The compositions of the present invention may contain from about 0.1 weight % to about 20 weight % waxes, based upon the total weight of the composition.

The gums are generally high molecular weight polydimethylsiloxanes (PDMSs), cellulose gums or polysaccharides, and the semi-solid materials are generally hydrocarbon-based compounds, such as, but not limited to, lanolins and derivatives thereof, or alternatively PDMSs The compositions of the present invention may contain from about 0.1 weight % to about 20 weight % gums, based upon the total weight of the composition, typically from about 0.5 weight % to about 10 weight %.

More particularly, the compositions for topical application can be in the form of a protective care composition for the skin, preferably for the face, the neck, the hands, the feet, or other areas of the body. Non-limiting examples include day creams or lotions, night creams or lotions, moisturizer, salves, sunscreen creams, lotions, or oils, ointments, gels, body milks, makeup (a foundation, a bronzer), artificial tanning compositions, depilatories, patches, emulsifiers, or a solid which is poured or cast as a stick or a dish, for example. The system of cosmetic compositions is ideal for use in a two-step foundation product because it may achieve high camouflage and blurring effects to result in the perception of a natural appearance.

In another embodiment, the topical compositions of the present invention may also include one or more of the following: a skin penetration enhancer, an absorbant, a sunscreen, an emollient, a skin plumper, an optical diffuser, a sunscreen, an exfoliation promoter, and an antioxidant. Details with respect to these and other suitable cosmetic ingredients can be found in the international Cosmetic Ingredient Dictionary (ICID) and Handbook, 10th Edition (2004), published by the Cosmetic, Toiletry, and Fragrance Association (CTFA), at pp. 2177-2299, which is herein incorporated by reference in its entirety.

An emollient provides the functional benefits of enhancing skin smoothness, reducing the appearance of fine lines and coarse wrinkles, and moisturizing. Non-limiting examples include isopropyl myristate, petrolatum, isopropyl lanolate, silicones (e.g., methicone, dimethicone), oils, mineral oils, fatty acid esters, or any mixtures thereof. The emollient is preferably present from about 0.1 weight % to about 50 weight % of the total weight of basecoat.

A skin plumper serves as a collagen enhancer to the skin. An example of a suitable, and preferred, skin plumper is palmitoyl oligopeptide. Other skin plumpers are collagen and/or glycosaminoglycan (GAG) enhancing agents. The skin plumper is preferably present from about 0.1 weight % to about 20 weight % of the total weight of the basecoat.

A sunscreen protects the skin from damaging ultraviolet rays. In an illustrative embodiment of the invention, the sunscreen would provide both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, non-mesoporous titanium dioxide, zinc oxide, or any mixtures thereof. The sunscreen may be present from about 1 weight % to about 30 weight % of the total weight of the composition. The addition of a sunscreen may protect the skin from ultraviolet radiation. The compositions of the invention having sunscreen bring about additional improvements to the aesthetic appearance of skin, including at least one of the following: minimizes sunburning, minimizes tanning, and reduces redness.

In an embodiment of the invention, one or more compositions may also have one or more exfoliation promoters. Suitable examples of an exfoliation promoter that can be used in the present compositions include alpha hydroxy acids (AHA); benzoyl peroxide; beta hydroxy acids; keto acids, such as pyruvic acid, 2-oxopropanoic acid, 2-oxobutanoic acid, and 2-oxopentanoic acid; oxa acids as disclosed in U.S. Pat. Nos. 5.847.003 and 5,834,513 (the disclosures of which are incorporated herein by reference); salicylic acid; urea; or any mixtures thereof. Preferred exfoliation promoters include 3,6,9-trioxaundecanedioic acid, glycolic acid, lactic acid, or any mixtures thereof. (See also, ICID at p. 2205).

When an embodiment of the invention includes an exfoliation promoter, the composition has about 0.1 weight % to 30 weight %, preferably about 1.0 weight % to about 1.5 weight % and more preferably about 1.0 weight % to about 10 weight %, of the exfoliation promoter based on the total weight of the composition.

An antioxidant functions, among other things, to scavenge free radicals from skin to protect the skin from environmental aggressors. Examples of antioxidants that may be used in the present compositions include compounds having phenolic hydroxy functions, such as ascorbic acid and its derivatives/esters; beta-carotene; catechins; curcumin; ferulic acid derivatives (e.g. ethyl ferulate, sodium ferulate); gallic acid derivatives (e.g. propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (—SH), in either reduced or non-reduced form, such as glutathione, lipose acid, thioglycolic acid, and other sulfhydryl compounds. The antioxidant may be inorganic, such as bisulfites, metabisulfites, sulfites, or other inorganic salts and acids containing sulfur. Compositions of the present invention may have an antioxidant preferably from about 0.001 weight % to about 10 weight %, and more preferably from about 0.01 weight % to about 5 weight %, of the total weight of the composition. (See also, ICID at p. 2184).

In one embodiment of the invention, one or more compositions may also have one or more of the following cosmetic and pharmaceutical active agents, excipients, ingredients, or adjuvants; anesthetics, antibiotics (e.g., erythromycins and tetracyclines) salicylic acids, anti-allergenics, antifungals, antiseptics, anti-irritants, anti-inflammatory agents, antimicrobials, analgesics, nitric oxide synthase inhibitors, insect repellents, self-tanning agents, skin penetration enhancers, skin cooling agents, chelating agents, colorants including dyes, lakes and pigments that may be untreated or chemically surface treated to improve wetability or some other property, demulcents, emollients, emulsifiers, fragrances, humectants, lubricants, skin protectants, moisturizers, pH adjusters, preservatives, stabilizers, surfactants, thickeners, film formers, plasticizers, viscosity modifiers, vitamins, or any mixtures thereof. The amounts of these various substances are those that are conventionally used in the cosmetic or pharmaceutical fields to achieve their intended purposes, for example, they may constitute from about 0.01% to 20% of the total weight of a composition.

Non-limiting examples of active agents for formulating into one or more compositions of the invention include those reagents having an effect on the treatment of wrinkles and/or fine lines, in addition to the actives as described, such as keratolytic agents, i.e., an active agent having desquamating, exfoliant, or scrubbing properties, or an active agent which can soften the horny layer of the skin. Other examples of anti-wrinkle or anti-fine line active agents include hydroxy acids and retinoids. These agents can be formulated, for example, in amounts of from about 0.01% to 5% by weight relative to the total weight of a composition.

Suitable hydroxy acids include, for example, glycoiic acid, lactic acid, malic acid, tartaric acid, citric acid, 2-hydroxyalkanoic acid, mandelic acid, salicylic acid and alkyl derivatives thereof including 5-n-octanoylsalicylic acid, 5-n-dodecanoylsalicylic acid, 5-n-decanoylsalicylic acid, 5-n-octylsalicylic acid, 5-n-heptyloxysalicylic acid, 4-n-heptyloxysalicylic acid and 2-hydroxy-3-methylbenzoic acid or alkoxy derivatives thereof, such as 2-hydroxy-3-methyoxybenzoic acid.

Emulsifiers are typically present in one or more compositions of the invention in an amount of about 0.0.1 weight % to 30 weight %, by weight and preferably from about 0.1 weight % to 30 weight % by weight relative to the total weight of the composition. However, not all compositions will necessarily include emulsifiers. (See e.g., ICID at p. 2276-2285).

Non-limiting examples of suitable thickening agents include xanthan gum, hydroxypropyl cellulose, hydroxyethyl cellulose, carbomer, gum acacia, Sepigel 305 (available from Seppic Co., France), and clays such as magnesium aluminum silicate. (See, e.g., ICID at p. 2293-2299).

Topical compositions of the present invention may include, and their utility can be enhanced by, one or more humectants, such as ureas, pyrrolidone carboxylic acids, amino acids, sodium hyaluronates, certain polyols and other compounds with hygroscopic properties. (See ICID at p. 2244).

The general activity and mildness to skin of the present topical compositions can also be enhanced by neutralization to pH about 3.5 to about 7.0, most preferably from pH about 3.7 to about 5.6. This neutralization is preferably accomplished with one or more of ammonium hydroxide, potassium hydroxide, sodium hydroxide, arginine or other amino acids, and/or triethanolamine.

Exemplary retinoids include, without limitation, retinoic acid (e.g., all-trans or 13-cis) and derivatives thereof retinol (Vitamin A) and esters thereof, such as retinol palmitate, retinol acetate and retinol propionate, and salts thereof.

One or more compositions of the present invention may be formulated with a cosmetically or demagogically acceptable vehicle, medium, diluent or carrier. The one or more compositions may be further formulated according to procedures known in the art to provide cosmetic compositions such as emulsions, gels, creams, lotions, masks, toners, serums, oils, water-in-oil, oil-in-water, water-oil-water triple emulsions having the appearance of a cream or gel, microemulsions, ointments, pastes, sticks, cakes, pencils, aerosol, and essences, as well as other topical cosmetic vehicles, it is also contemplated that topical compositions of the present invention can be incorporated into delivery systems such as liposomes and topical patches, tapes, and sprays.

In addition, the compositions may be in the form of vesicular dispersions containing ionic and/or nonionic lipids, as described above. Dosage units suitable for such compositions are formulated according to the conventional knowledge and techniques used in the art.

The following example describes specific aspects of the invention to illustrate the invention and provide a description of the present methods for those skilled in the art. The example should not be construed as limiting the invention, as the example merely provides specific methodology useful in the understanding and practice of the invention and its various aspects.

### EXAMPLE

In this example, a system of cosmetic compositions is prepared and tested to measure diffused transmittance and reflectance. The system includes two compositions, a pigmented basecoat and a diffused topcoat. The pigmented basecoat includes titanium dioxide, and is applied to clean glass slides in three (3) thicknesses: 2 microns, 5 microns, and 10 microns. The diffused topcoat is applied to the basecoat. The diffused topcoat includes nylon. The diffused topcoat is applied at a thickness of 10 microns.

At a basecoat thickness of 2 microns, the pigmented basecoat and diffused topcoat exhibit a percent diffused transmittance of about 30. At a basecoat thickness of 5 microns, the pigmented basecoat and diffused topcoat exhibit a percent diffused transmittance of about 15. At a basecoat thickness of 10 microns, the pigmented basecoat and diffused topcoat exhibit a percent diffused transmittance of about 9. These results are reported in FIG. 4.

Further, at a basecoat thickness of 2 microns, the pigmented basecoat and diffused topcoat exhibit a percent reflectance of about 40. At a basecoat thickness of 5 microns, the pigmented basecoat and diffused topcoat exhibit a percent diffused transmittance of about 69. At a basecoat thickness of 10 microns, the pigmented basecoat and diffused topcoat exhibit a percent diffused transmittance of about 83. These results are reported in FIG. 5.

The content of all patents, patent applications, published articles, abstracts, books, reference manuals and abstracts, as cited herein are hereby incorporated by reference in their entireties to more fully describe the state of the art to which the invention pertains. All concentrations recited in the specification and claims are reported as weight percents, unless otherwise indicated.

It should be understood that the foregoing description is only illustrative of the present invention. Various alternatives and modifications can be devised by those skilled in the art without departing from the invention. Accordingly, the present invention is intended to embrace all such alternatives, modifications and variations that fall within the scope of the appended claims.

What is claimed is:

1. A cosmetic kit comprising:
(a) a first composition comprising
(1) a pigment, and
(2) a cosmetically or pharmaceutically acceptable vehicle; and
(b) a second composition comprising
(1) a diffuser,
(2) a film former, and
(3) a cosmetically or pharmaceutically acceptable vehicle.

2. A kit of claim 1 wherein said diffuser comprises nylon and said pigment comprises titanium dioxide.

3. A kit of claim 1 wherein the first composition has an index of refraction of between about 1.38 and about 3.52 and wherein the second composition has an index of refraction of between about 1.30 to about 2.20.

4. A kit of claim 1 wherein the first composition has a first index of refraction and the second composition has a second index of refraction, and wherein the difference between said first index of refraction and said second index of refraction is about 0.01 to about 1.50.

5. A kit of claim 1 wherein the first composition has an index of refraction of between about 1.42 to about 1.60 and the second composition has an index of refraction of about 1.40 to about 2.00.

6. A kit of claim 1 wherein said pigment is selected from the group consisting of titanium dioxide, zirconium oxide, cerium oxide, zinc oxide, iron oxide, chromium oxide, ferric blue, barium, strontium, calcium, aluminum lakes and carbon black.

7. A kit of claim 6 wherein said diffuser is selected from the group consisting of silica, fumed silica, polyurethane, poly(methyl methacrylate), nylon, polyethylene, polystyrene, polytetrafluoroethylene, polymethylsiloxane, boron nitride, mica, fumed alumina and cellulosics.

8. A kit of claim 1 wherein said pigment comprises titanium dioxide and said diffuser comprises silica.

9. A kit of claim 1 wherein said pigment comprises iron oxide.

10. A method of using a first cosmetic composition and a second cosmetic composition, said first cosmetic composition comprising a pigment and a cosmetically or pharmaceutically acceptable vehicle, and said second cosmetic composition comprising a diffuser, a film former, and a cosmetically or pharmaceutically acceptable vehicle, said method comprising applying a basecoat of said first cosmetic composition to skin and then applying a topcoat of said second cosmetic composition to said basecoat, wherein said first cosmetic composition and said second cosmetic composition are applied in amounts for improving the appearance of skin.

11. A method of claim 10 wherein said pigment comprises titanium dioxide and said diffuser comprises nylon.

12. A method of claim 10 wherein said basecoat has a thickness of between approximately 1 and approximately 20 microns and said topcoat has a thickness of between approximately 1 and approximately 20 microns.

13. A method of claim 10 wherein said second cosmetic composition has a percent reflectance of about 60 or less.

14. A method of claim 12 wherein said second cosmetic composition has a percent reflectance of about 40 or less, and wherein said pigment comprises titanium dioxide and said diffuser comprises nylon.

15. A method of claim 12 wherein said second cosmetic composition has a percent reflectance of about 20 or less.

16. A method of applying cosmetic compositions to a biological surface, the method comprising first
(a) applying to the biological surface a basecoat of a first composition comprising
(1) a pigment and
(2) a cosmetically or pharmaceutically acceptable vehicle, and subsequently
(b) applying to the basecoat a topcoat, of a second composition comprising
(1) a diffuser,
(2) a film former, and
(3) a cosmetically or pharmaceutically acceptable vehicle.

17. A method of claim 16, wherein said diffuser comprises nylon and said pigment comprises titanium dioxide.

18. A method of claim 16, wherein said first composition has an index of refraction of between about 1.42 and about 1.60 and said second composition has an index of refraction of between about 1.40 to about 2.00.

19. A method of claim 16, wherein said basecoat has a thickness of between approximately 1 and approximately 20 microns and said topcoat has a thickness of between approximately 1 and approximately 20 microns.

20. A method of claim 19, wherein said second composition has a percent reflectance of about 40 or less, and wherein said pigment comprises titanium dioxide and said diffuser comprises nylon.

21. A method of claim 19 wherein said pigment comprises iron oxide.

22. A method of claim 19 wherein said second composition has a percent reflectance of about 20 or less.

23. A layered arrangement of cosmetic compositions, said arrangement comprising a substrate, a first composition, and a second composition such that the first composition is formed as a basecoat contacting the substrate and the second composition is formed as a topcoat contacting the first composition, said first composition comprising a pigment and said second composition comprising a diffuser.

24. A layered arrangement of cosmetic compositions of claim 23, wherein said topcoat is approximately 10 microns in thickness or less.

25. A layered arrangement of cosmetic compositions of claim 24, wherein said topcoat has a percent reflectance value of about 60 or less.

26. A layered arrangement of cosmetic compositions of claim 24, wherein said topcoat has a percent reflectance value of about 20 or less.

27. A layered arrangement of cosmetic compositions of claim 23, wherein said pigment comprises titanium dioxide and said diffuser comprises nylon.

28. A layered arrangement of cosmetic compositions of claim 23 having a percent reflectance value of about 10 or greater.

29. A layered arrangement, of cosmetic compositions of claim 23 having a percent reflectance value of about 20.

30. A layered arrangement of cosmetic compositions of claim 23 having a percent reflectance value of about 25.

31. A layered arrangement of cosmetic compositions of claim 23 having a percent reflectance value of about 75.

32. A layered arrangement of cosmetic compositions of claim 23 wherein said first composition comprises iron oxide.
